(19) United States
(12) Patent Application Publication
(10) Pub. No.: US 2015/0359589 A1
(43) Pub. Date: Dec. 17, 2015

(54) INTRAVASCULAR NEUROMODULATION DEVICE HAVING A HELICAL THERAPEUTIC ASSEMBLY WITH PROUD PORTIONS AND ASSOCIATED METHODS

(71) Applicant: Medtronic Ardian Luxembourg S.a.r.l., Luxembourg (LU)

(72) Inventors: Kevin Mauch, Windsor, CA (US); Martin Rothman, Santa Rosa, CA (US)

(21) Appl. No.: 14/301,508

(22) Filed: Jun. 11, 2014

(51) Int. Cl. A61B 18/14; A61M 25/09; A61M 25/00; A61N 1/05; A61N 1/36

(52) U.S. Cl. CPC A61B 18/1492; A61N 1/05; A61N 1/36117; A61M 25/0045; A61M 25/09; A61B 2018/1407; A61B 2018/00511

(57) ABSTRACT

Catheter apparatuses, systems, and methods for achieving neuromodulation by intravascular access. A treatment device has a pre-formed helical therapeutic assembly with spaced-apart proud portions that are offset with respect to the pre-formed helical shape when in a deployed configuration. The therapeutic assembly includes a plurality of energy delivery elements carried by and associated with the proud portions such that, in the deployed configuration, the proud portions are configured to position the energy delivery elements in apposition with an inner wall of a target blood vessel. The energy delivery elements can deliver energy across the inner wall of a renal artery, for example, to heat or otherwise electrically modulate neural fibers that contribute to renal function.

### TECHNICAL FIELD

The present technology relates generally to intravascular neuromodulation and associated methods. In particular, several embodiments are directed to devices having generally helix-shaped support structures with spaced-apart proud portions for intravascular renal neuromodulation and associated methods.

### BACKGROUND

The sympathetic nervous system (SNS) is a primarily involuntary bodily control system typically associated with stress responses. Fibers of the SNS innervate tissue in almost every organ system of the human body and can affect characteristics such as pupil diameter, gut motility, and urinary output. Such regulation can have adaptive utility in maintaining homeostasis or preparing the body for rapid response to environmental factors. Chronic activation of the SNS, however, is a common maladaptive response that can drive the progression of many disease states. Excessive activation of the renal SNS in particular has been identified experimentally and in humans as a likely contributor to the complex pathophysiology of hypertension, states of volume overload (such as heart failure), and progressive renal disease. For example, radiotracer dilution has demonstrated increased renal norepinephrine (“NE”) spillover rates in patients with essential hypertension.

Cardio-renal sympathetic nerve hyperactivity can be particularly pronounced in patients with heart failure. For example, an exaggerated NE overflow from the heart and kidneys of plasma is often found in these patients. Heightened SNS activation commonly characterizes both chronic and end stage renal disease. In patients with end stage renal disease, NE plasma levels above the median have been demonstrated to be predictive of cardiovascular diseases and several causes of death. This is also true for patients suffering from diabetic or contrast nephropathy. Evidence suggests that sensory afferent signals originating from diseased kidneys are major contributors to initiating and sustaining elevated central sympathetic outflow.

Sympathetic nerves innervating the kidneys terminate in the blood vessels, the juxtaglomerular apparatus, and the renal tubules. Stimulation of the renal sympathetic nerves can cause increased renin release, increased sodium (Na+) reabsorption, and a reduction of renal blood flow. These neural regulation components of renal function are considerably stimulated in disease states characterized by heightened sympathetic tone and likely contribute to increased blood pressure in hypertensive patients. The reduction of renal blood flow and glomerular filtration rate as a result of renal sympathetic efferent stimulation is likely a cornerstone of the loss of renal function in cardio-renal syndrome (i.e., renal dysfunction as a progressive complication of chronic heart failure). Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). These pharmacologic strategies, however, have significant limitations including limited efficacy, compliance issues, side effects, and others. Recently, intravascular devices that reduce sympathetic nerve activity by applying an energy field to a target site in the renal blood vessel (e.g., via radio frequency ablation) have been shown to reduce blood pressure in patients with treatment-resistant hypertension.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. Furthermore, components can be shown as transparent in certain views for clarity of illustration only and not to indicate that the illustrated component is necessarily transparent. For ease of reference, throughout this disclosure identical reference numbers may be used to identify identical or at least generally similar, analogous and/or complementary components or features.

FIG. 1 is a partially schematic diagram of a neuromodulation system configured in accordance with an embodiment of the present technology.

FIG. 2 is a longitudinal cross-sectional view of an intravascular therapeutic assembly in a delivery configuration (e.g., low-profile or collapsed configuration) and carried within a delivery element in accordance with an embodiment of the present technology.

FIG. 3A is a perspective view of the distal portion of the therapeutic assembly of FIG. 2 in a deployed configuration (e.g., expanded configuration) in accordance with an embodiment of the present technology.

FIG. 3B is a side view of the intravascular therapeutic assembly of FIG. 2 having a spiral-shaped support structure in a deployed configuration (e.g., expanded configuration) within a renal artery of a patient in accordance with a further embodiment of the present technology.

FIG. 3C is a transverse cross-sectional view of the intravascular therapeutic assembly taken along line 3C-3C of FIG. 3B.

FIG. 3D is an enlarged, partially cross-sectional view of a portion of the therapeutic assembly of FIG. 3A.

FIG. 4 is a longitudinal cross-sectional view of an intravascular therapeutic assembly in a delivery configuration (e.g., low-profile or collapsed configuration) and carried within a delivery element in accordance with an embodiment of the present technology.

FIG. 5A illustrates a treatment device configured in accordance with an embodiment of the present technology in a delivery configuration (e.g., low-profile or collapsed configuration) outside a patient.

FIG. 5B illustrates the treatment device of FIG. 5A in a deployed configuration (e.g., expanded configuration) outside a patient.

FIG. 6 schematically illustrates modulating renal nerves with an intravascular therapeutic assembly configured in accordance with an embodiment of the present technology.

### DETAILED DESCRIPTION

The present technology is directed to apparatuses, and methods for achieving electrically- and/or thermally-induced renal neuromodulation (i.e., rendering neural fibers that innervate the kidney inert, inactive or otherwise completely or partially reduced in function) by percutaneous transluminal intravascular access. In particular, embodiments of the present technology relate to treatment devices (e.g., treatment catheters) having therapeutic assemblies with support structures that provide a pre-formed, generally helical shape with spaced-apart proud portions, such as steps, platforms or other structures protruding from the neighboring portions of the support structure. The therapeutic assemblies include neuromodulation elements (e.g., energy delivery elements, band electrodes, etc.) that can be associated, for example, with the proud portions of the treatment device. After being positioned in a target blood vessel of a human patient, a therapeutic assembly is transformable between a delivery configuration having a low-profile configured to pass through the vasculature and a deployed configuration in which the therapeutic assembly has a radially expanded shape (e.g., generally spiral/helical or coil) and in which the proud portions or steps maintain stable apposition between the neuromodulation elements and an inner wall of the target blood vessel (e.g., renal artery). Although it is the shape of the pre-formed support structure that tends to dominate or define the shape of the therapeutic assembly in the deployed configuration, other components of the assembly may also contribute to the shape of the deployed configuration. Therefore, the term “deployed configuration” can refer to the treatment device, the therapeutic assembly, the support structure, or other components that are actively or passively involved in the transformation between the delivery configuration and the deployed configuration.

The treatment devices may also be part of a system that can also include an energy source or energy generator external to the patient in electrical communication with the neuromodulation element(s). In operation, the neuromodulation element(s) are advanced intravascularly to a target blood vessel, such as the renal artery, along a percutaneous transluminal path (e.g., a femoral artery puncture, an iliac artery and the aorta, a radial artery, or another suitable intravascular path), and then energy is delivered to the wall of the target blood vessel via the neuromodulation element(s). Suitable energy modalities include, for example, electrical energy, radio frequency (RF) energy, pulsed electrical energy, or thermal energy. The treatment device carrying the neuromodulation element(s) can be configured such that the neuromodulation element(s) are in steady apposition with the interior wall of the target blood vessel when the therapeutic assembly is in the deployed configuration, e.g., radially expanded to have a spiral/helical shape. The proud portions or steps are offset with respect to adjacent and/or interposing regions of the support structure when the support structure is in the spiral/helical deployed configuration. The proud portions, for example, can protrude radially outward relative to neighboring portions of the support structure to contact the inner wall of the target blood vessel such that interposing segments of the support structure may have reduced contact force with, or be spaced radially inward and apart from the inner wall of the target blood vessel. The pre-formed spiral/helical shape of the deployed therapeutic assembly allows blood to flow through the assembly during therapy, which is expected to help prevent occlusion of the blood vessel during activation of the neuromodulation element(s), while the proud portions offset from the spiral/helical shape provide unobstructed or focused contact regions for the neuromodulation elements to enhance apposition with the inner wall of the target blood vessel.

Known energy-delivery catheter systems for inducing neuromodulation include one or more electrodes mounted on a positioning element, e.g. a balloon, a basket or a helical shaft that can itself contact the inner wall of the blood vessel and, in doing so, may compromise the desired contact between electrodes and the inner wall. For example, a portion of a positioning element near an electrode may contact an irregular surface of the interior wall of the blood vessel and thereby impair the integrity of or even prevent the contact between the electrode and the vessel wall. This can cause the measured impedance to be higher at such an electrode and result in an inconsistent lesion being formed on the interior wall of the blood vessel.

Several embodiments of the present technology have a support structure with proud portions that are offset radially outward with respect to adjacent and/or intervening portions of the support structure between the proud portions when the support structure is in a helical shape upon deployment. As such, neuromodulation elements (e.g., energy delivery elements, electrodes, etc.) mounted at the proud portions may more assuredly contact the interior wall of the blood vessel. In various embodiments, the offset of the proud portions may space the adjacent and/or interposing portions of the support structure inwardly apart from the interior wall. This reduced vessel wall contact by non-electrode portions of the therapeutic assembly is expected to increase the consistency of the electrical impedance measured between the neuromodulation elements and the surface of the interior wall of the blood vessel and thereby cause more consistent lesions to be produced as compared to conventional positioning elements that lack proud portions. For example, reliable radial and longitudinal contact of the electrodes with the inner wall of the target blood vessel may provide benefits, such as more reliable energy transmission, which may lower energy requirements and improve the accuracy of impedance and temperature measured at the inner wall of the target blood vessel. Therapeutic assemblies of the present technology also have a low-profile, collapsed delivery configuration in which the proud portions and associated neuromodulation elements are at least approximately in axial alignment with the longitudinal axis of the intervening portions of the low-profile support structure. As such, the present design allows for delivery of the treatment device through the vasculature in a low-profile guide catheter or delivery sheath.

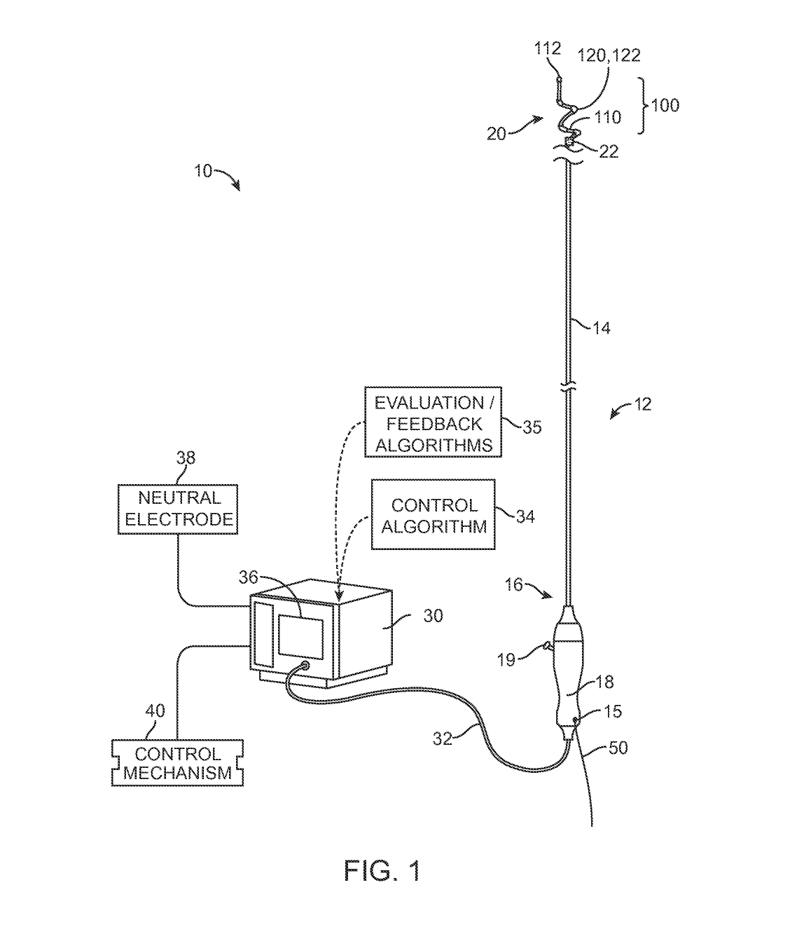

Specific details of several embodiments of the technology are described below with reference to FIGS. 1-6. Although many of the embodiments are described below with respect to devices, systems, and methods for intravascular modulation of renal nerves using helix-shaped support structures, other applications and other embodiments in addition to those described herein are within the scope of the technology. Additionally, several other embodiments of the technology can have configurations, components, or procedures that differ from those described herein. A person of ordinary skill in the art, therefore, will accordingly understand that the technology can have other embodiments with additional elements, or the technology can have other embodiments without several of the features shown and described below with reference to FIGS. 1-6.

As used herein, the terms “distal” and “proximal” define a position or direction with respect to the treating clinician or clinician's control device (e.g., a handle assembly). “Distal” or “distally” are a position distant from or in a direction away from the clinician or clinician's control device. “Proximal” and “proximally” are a position near or in a direction toward the clinician or clinician's control device.

### Selected Examples of Neuromodulation Systems

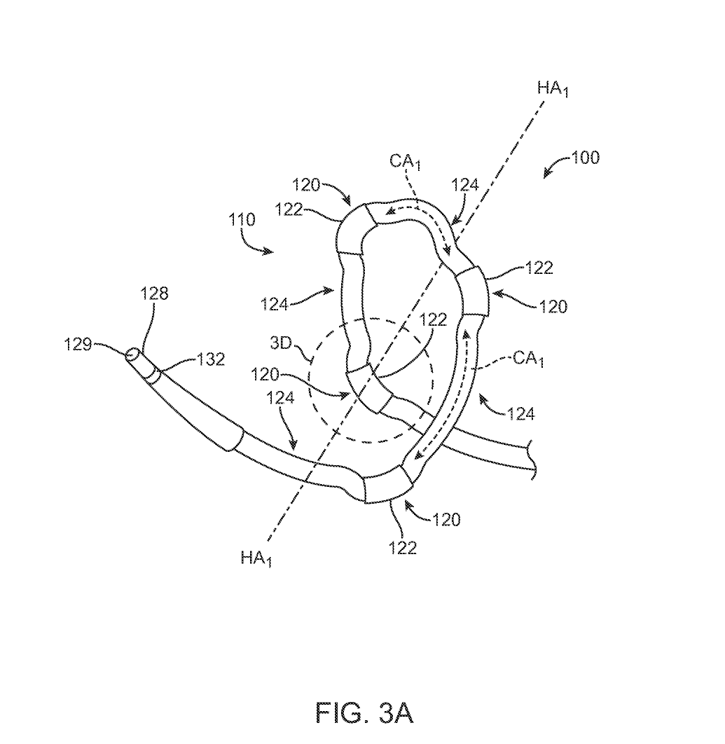

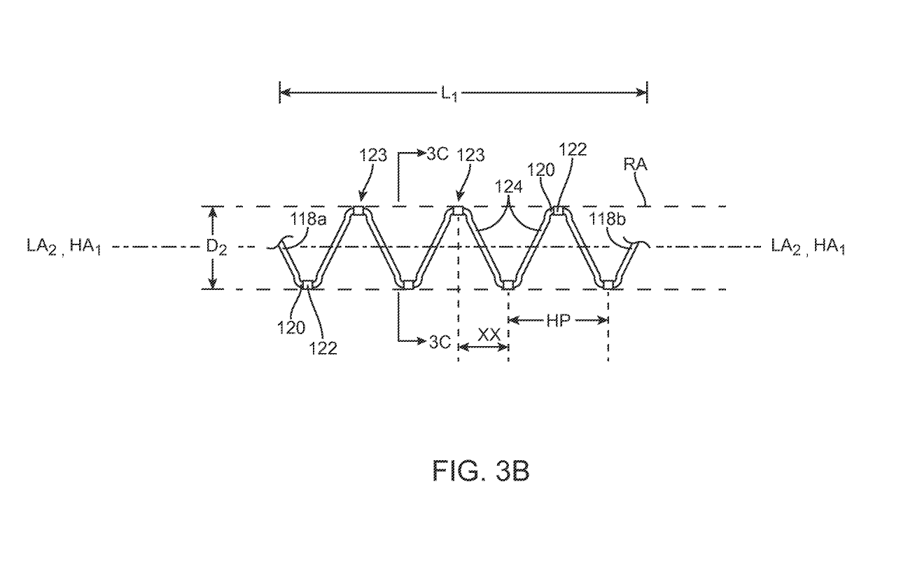

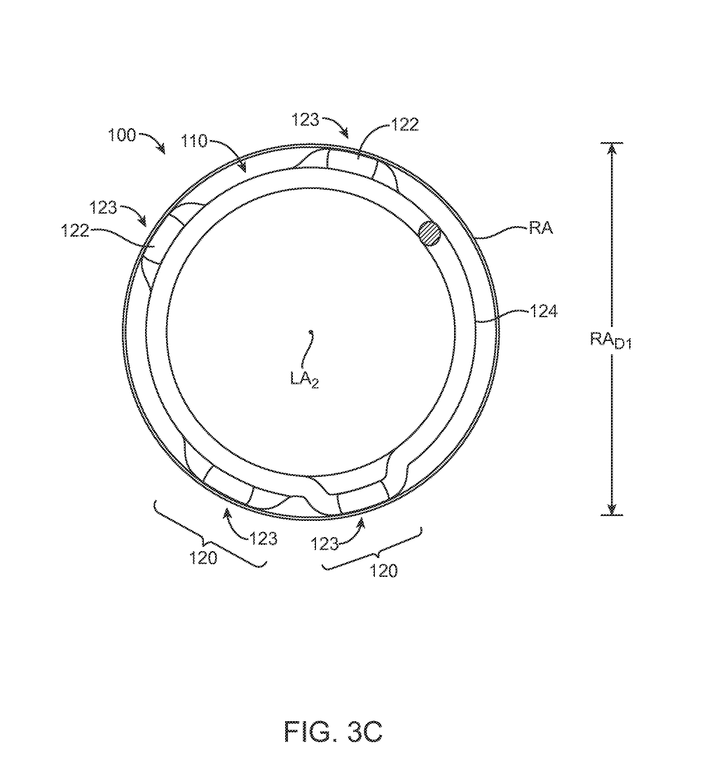

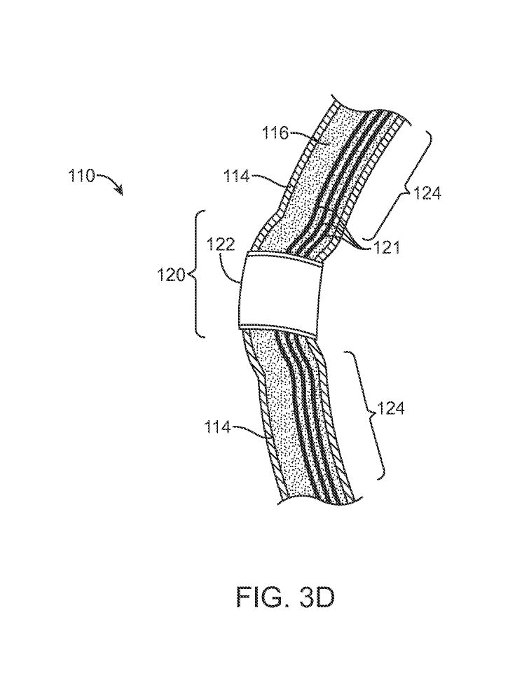

FIG. 1 is a partially schematic illustration of a renal neuromodulation system 10 (“system 10”) configured in accordance with an embodiment of the present technology. System 10 includes an intravascular catheter 12 and an energy source or energy generator 30 (e.g., a RF energy generator) operably coupled to the catheter 12. Catheter 12 can include an elongated shaft 14 having a proximal portion 16 and a distal portion 20. Catheter 12 also includes a handle 18 mounted at proximal portion 16. Catheter 12 can further include a therapeutic assembly 100 (shown schematically), such as a treatment section, at the distal portion 20 (e.g., attached to distal portion 20, defining a section of distal portion 20, etc.). As explained in further detail below, therapeutic assembly 100 can include a support structure 110 and an array of one or more neuromodulation elements 122 at areas along the support structure 110. Therapeutic assembly 100 is transformable into a delivery configuration having a low-profile to navigate a patient's vasculature and position neuromodulation elements 122 in a renal blood vessel (e.g., a renal artery). Upon delivery to the target treatment site within the renal blood vessel, therapeutic assembly 100 is transformable into a deployed configuration having an expanded, generally spiral/helical shape for apposing neuromodulation elements 122 against the inner wall of the blood vessel for delivering energy at the treatment site and providing therapeutically-effective electrically- and/or thermally-induced renal neuromodulation. As shown in FIG. 3A the pre-formed helical shape of the deployed configuration defines a curvilinear axis CA₁ about a central helical axis HA₁. Support structure 110 has a plurality of proud portions 120 (e.g., steps, platforms, etc.; shown in FIG. 3C) that are spaced apart by adjacent or interposing portions 124 and are offset from a curvilinear axis CA₁ of the support structure in a direction radially outward from central helical axis HA₁ to press neuromodulation elements 122 against an inner wall of the renal artery RA. In other words, proud portions 120 are not aligned (e.g., not collinear and/or not concentrically centered) with curvilinear axis CA₁ of support structure 110. In an alternative embodiment, therapeutic assembly 100 may be non-spiral/non-helical in the deployed configuration provided that therapeutic assembly 100 delivers the energy to the treatment site.

Therapeutic assembly 100 may be transformed between the delivery and deployed configurations using a variety of suitable mechanisms or techniques (e.g., self-expansion). In one specific example, support structure 110 can include a pre-formed, self-expanding tubular structure that tends to take on the deployed configuration when unconstrained (e.g., by retracting a guidewire, a guide catheter, straightening sheath, etc.). FIG. 1 illustrates a proximal end of a guidewire 50 extending from an exit port 15 in handle 18 and an actuator 19, such as a knob, pin, or lever carried by handle 18. Guidewire 50 and/or actuator 19 or other suitable mechanisms or techniques may be provided for transforming therapeutic assembly 100 between the delivery and deployed configurations.

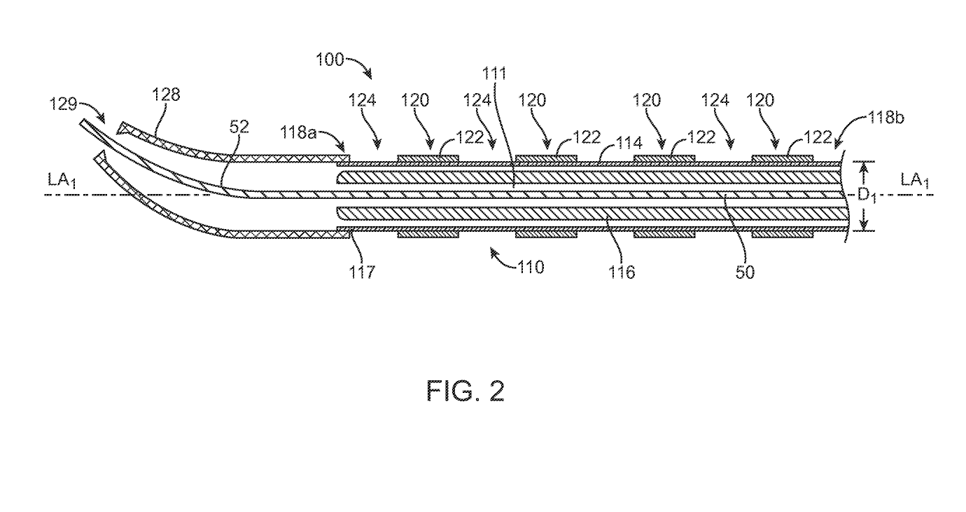

The proximal end of support structure 110 is carried by or affixed to distal portion 20 of elongated shaft 14. Catheter 12 may also include an atraumatic tip 112 at the distal end of support structure 110 to prevent intravascular trauma during delivery of the therapeutic assembly 100 to the treatment site. The distal end of catheter 12 may also be configured to engage another element of system 10 or catheter 12. For example, the distal end of catheter 12 may define a passageway for receiving guidewire 50 for delivery of the treatment device using over-the-wire (“OTW”) or rapid exchange (“RX”) techniques. Further details regarding such arrangements are described below with reference to FIG. 2.

Neuromodulation element(s) 122 can be electrically coupled to energy source 30 via a cable 32, and energy source 30 (e.g., an RF energy generator) can be configured to produce a selected modality and magnitude of energy for delivery to the treatment site via neuromodulation elements 122 at proud portions 120 of support structure 110. As described in greater detail below, one or more supply wires (not shown) can extend along elongated shaft 14 or through a lumen in shaft 14 to therapeutic assembly 100 and supply the treatment energy to neuromodulation elements 122.

System 10 can further include a control mechanism 40, such as foot pedal or handheld remote control device, connected to energy source 30 to allow the clinician to initiate, terminate and, optionally, adjust various operational characteristics of energy source 30, including, but not limited to, power delivery. The remote control device 40 can be positioned in a sterile field and operably coupled to the therapeutic assembly 100, and specifically to neuromodulation elements 122, and can be configured to allow the clinician to activate and deactivate the energy delivery to neuromodulation elements 122. In other embodiments, the remote control device may be built into handle assembly 18.

The energy source or energy generator 30 can be configured to deliver the treatment energy via an automated control algorithm 34 and/or under the control of a clinician. For example, energy source 30 can include computing devices (e.g., personal computers, server computers, tablets, etc.) having processing circuitry (e.g., a microprocessor) that is configured to execute stored instructions relating to control algorithm 34. In addition, the processing circuitry may be configured to execute one or more evaluation/feedback algorithms 35, which can be communicated to the clinician. For example, energy source 30 can include a monitor or display 36 and/or associated features that are configured to provide visual, audio, or other indications of power levels, sensor data, and/or other feedback. Energy source 30 can also be configured to communicate the feedback and other information to another device, such as a monitor in a catheterization laboratory.

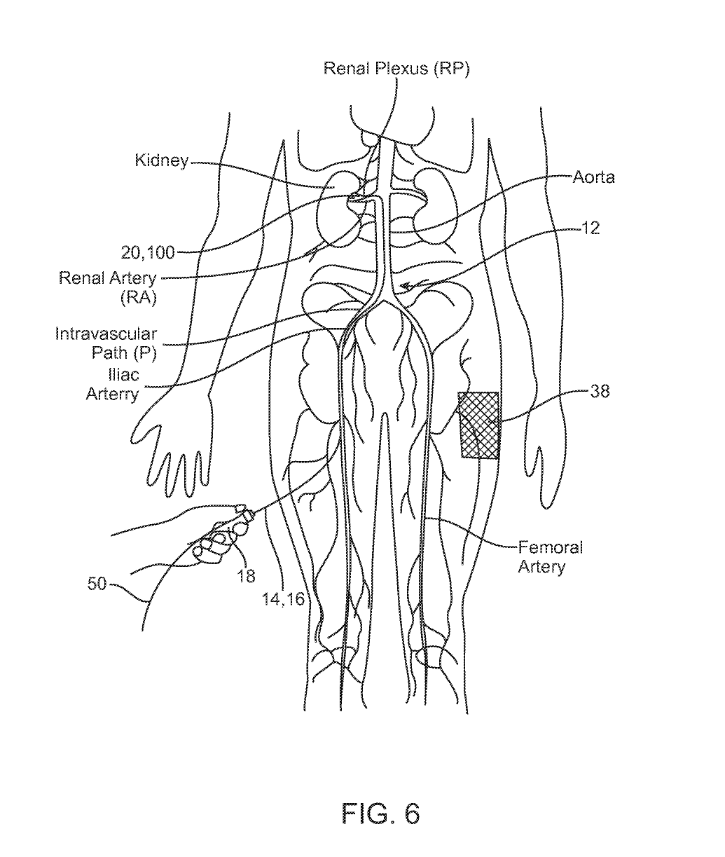

System 10 may be configured to provide monopolar or bipolar electric fields via neuromodulation elements 122. In embodiments configured to deliver monopolar electric fields, system 10 also includes a neutral or dispersive electrode 38 electrically connected to energy generator 30 and attached to the exterior of the patient, as shown in FIG. 6, to provide a return path for the electrical current delivered from neuromodulation elements 122. In embodiments configured to deliver bipolar electric fields, neuromodulation elements 122 are bipolar electrodes. Individual neuromodulation elements 122 are connected to energy generator 30 and are associated with proud portions 120 for directly contacting an internal wall of the artery (e.g., the renal artery). The application of RF electric field energy serves to ohmically or resistively heat tissue in the vicinity of the electrode and thereby thermally injures the heated tissue. The treatment objective is to thermally induce neuromodulation (e.g., necrosis, thermal alteration or ablation) in the targeted neural fibers. The thermal injury forms a lesion in the vessel wall. Alternatively, an RF electrical field may be delivered with an oscillating or pulsed intensity that does not thermally injure the tissue whereby neuromodulation in the targeted nerves is accomplished by electrical modification of the nerve signals.

System 10 can also include one or more sensors 22 located proximate to or within neuromodulation elements 122. For example, system 10 can include temperature sensors (e.g., thermocouples, thermistors, etc.), impedance sensors, pressure sensors, optical sensors, flow sensors, and/or other suitable sensors connected to one or more supply wires (not shown) that transmit signals from the sensors and/or convey energy to the therapeutic assembly 100.

### Selected Examples of Therapeutic Assemblies and Related Devices

FIG. 2 is a longitudinal cross-sectional view of a portion of intravascular therapeutic assembly 100 in a delivery configuration (e.g., a low-profile or collapsed configuration) in accordance with an embodiment of the present technology, and FIG. 3A is a perspective view of therapeutic assembly 100 of FIG. 2 in a deployed configuration (e.g., an expanded configuration). As noted above, therapeutic assembly 100 can be transformed or actuated between the delivery configuration (FIG. 2) and the deployed configuration (e.g., a radially expanded, generally spiral/helical configuration, FIG. 3A).

Referring to FIGS. 2 and 3A together, therapeutic assembly 100 includes support structure 110 which can include a flexible tube 114 and a pre-shaped spiral/helical control member 116 within tube 114, and a plurality of neuromodulation elements 122 spaced apart from each other along support structure 110. Therapeutic assembly 100 can define a tubular structure having a low-profile outer dimension D₁ (e.g., diameter of a circular or non-circular cross-section), a longitudinal axis LA₁, and a lumen 111 for slidably receiving guidewire 50 (FIG. 2). In the delivery configuration, proud portions 120 are at least approximately concentrically aligned with longitudinal axis LA₁ of the support structure 110; however, in the deployed configuration, for example, proud portions 120 are offset with respect to adjacent portions 124 when support structure 110 is in a pre-formed helical shape (FIG. 3A).

Referring to FIG. 2, one embodiment of therapeutic assembly 100 may be constrained in the delivery configuration by guidewire 50 disposed within lumen 111 of support structure 110. Guidewire 50 may be sufficiently stiff to keep support structure 110 relatively straight and proud portions 120 at least substantially aligned with support structure 110 in the delivery configuration. It will be understood that, without additional bending stiffness provided by either guidewire 50 or another delivery element (e.g., straightening sheath, guide catheter, etc.), support structure 110 will tend to take on the pre-formed shape of control member 116 (e.g., the helical shape with offset proud portions). The distalmost portion 52 of guidewire 50 is more flexible that the remainder of the guidewire. Thus, when guidewire 50 is at least partially withdrawn from therapeutic assembly 100, as illustrated in FIG. 3A, control member 116 provides a shape-recovery force sufficient to overcome the straightening force provided by distalmost portion 52 of guidewire 50 such that support structure 110 can deploy towards its spiral/helical shaped configuration with the proud portions 120 offset from curvilinear axis CA₁. Further, because distalmost portion 52 of guidewire 50 can remain at least partially within the therapeutic assembly 100 without deforming, e.g., straightening the deployed configuration (e.g., FIG. 3A), guidewire 50 can impart additional stability to the spiral-shaped therapeutic assembly 100 during treatment. This feature is expected to help mitigate or reduce problems associated with keeping the therapeutic assembly 100 in place during treatment (e.g., help with vasoconstriction).

In an alternate method step, guidewire 50 including distalmost portion 52 may be withdrawn completely from therapeutic assembly 100 to permit transformation of therapeutic assembly 100 into the deployed configuration while guidewire 50 remains within shaft 14. In yet another method step, guidewire 50 may be withdrawn completely from catheter 12. In any of the foregoing examples, the clinician can withdraw guidewire 50 sufficiently to observe transformation of therapeutic assembly 100 to the deployed configuration and/or until an X-ray image shows that distalmost portion 52 of guidewire 50 is at a desired location relative to therapeutic assembly 100 (e.g., at least partially withdrawn from the therapeutic assembly). In some methods, the extent of withdrawal of guidewire 50 can be based, at least in part, on the clinician's judgment with respect to the selected guidewire and the extent of withdrawal necessary to achieve deployment of the therapeutic assembly 100.

In one example, therapeutic assembly 100 terminates at an atraumatic tip 128 (FIGS. 2 and 3A). The atraumatic tip 128 can be a flexible straight or curved tip. In one embodiment, atraumatic tip 128 may have a distal opening 129 for accommodating guidewire 50. The curvature of tip 128 can be varied depending upon the particular sizing/configuration of therapeutic assembly 100. In some embodiments, tip 128 may also comprise one or more radiopaque markers 132 (FIG. 3A) and/or one or more sensors (not shown). In one embodiment, tip 128 can be part of support structure 110 (e.g., an extension of or integral with support structure 110). In one example, flexible tip 128 can be a more flexible tapered portion (e.g., about 5 to about 7 mm) of the distal end of support structure 110. Such an arrangement can be suitable for OTW delivery of therapeutic assembly 100 to the target treatment site. In another embodiment, tip 128 can be a separate component that may be affixed to the distal end 117 of support structure 110 via adhesive, crimping, over-molding, or other suitable techniques. Tip 128 can be made from a polymer material (e.g., a polyether block amide copolymer sold under the trademark PEBAX, or a thermoplastic polyether urethane resin sold under the trademarks ELASTHANE or PELLETHANE), or other suitable materials having the desired properties, including a selected durometer. In other embodiments, tip 128 may be formed from different material(s) and/or have a different arrangement.

FIG. 3B is a side view of therapeutic assembly 100 of FIG. 2 in a deployed configuration having an expanded configuration within renal artery RA (or other target blood vessel) of a patient. As noted above, therapeutic assembly 100 can be transformed or actuated between the delivery configuration (FIG. 2) and the deployed configuration (e.g., having a radially expanded, generally spiral/helical configuration, FIGS. 3A and 3B). Neuromodulation elements 122 depicted in FIGS. 2-3B are merely for illustrative purposes, and it will be appreciated that therapeutic assembly 100 can include a different number and/or arrangement of neuromodulation elements 122.

As best seen in FIG. 3B, after delivery to the target treatment site (e.g. renal artery RA), the distal portion of support structure 110 of therapeutic assembly 100 may be deployed to its expanded, helix-shaped configuration having spaced-apart proud portions 120. In one embodiment, for example, the distal portion of support structure 110 may be deployed by retracting guidewire 50 (e.g., FIG. 2) thereby allowing radial expansion of the pre-formed helical shape wherein pre-formed proud portions 120 are offset from adjacent portions 124 of deployed support structure 110 and extend radially outward from a central longitudinal axis LA₂ of renal artery RA. As shown in FIGS. 3A and 3B, proud portions 120 are offset from support structure 110 in a direction toward the inner wall of renal artery RA such that portions 120 provide discrete contact regions 123 between support structure 110 and the inner wall. The distal portion of support structure 110 having proud portions 120 can be configured to assume the deployed configuration when in an unconstrained condition. Guidewire 50 (FIG. 2), for example, can be pulled proximally while therapeutic assembly 100 is held stationary with respect to the treatment site. Alternatively, therapeutic assembly 100 can be pushed distally over or beyond distalmost portion 52 of guidewire 50 while the guidewire is held stationary with respect to the treatment site.

As shown in FIGS. 3A and 3B, support structure 110 can have a spiral/helix configuration characterized, at least in part, by its deployed or radially-expanded outer dimension, length, pitch (longitudinal distance of one complete helix turn measured parallel to central helical axis HA₁), and number of revolutions (number of times the helix completes a 360° revolution about the central spiral axis HA₁). FIG. 3B illustrates the deployed helical configuration of therapeutic assembly 100 dimensionally restricted by renal artery RA. As therapeutic assembly 100 deploys from its delivery configuration, its low-profile outer dimension D₁ (FIG. 2) increases to a vessel-limited outer dimension D₂. Catheter 12 is designed or selected to have a therapeutic assembly 100 that tends to deploy in free space to an outer diameter or range of outer diameters larger than the diameter of the target site in the patient's anatomy. The radial restraint of therapeutic assembly 100 by, i.e., renal artery RA generates a contact force between neuromodulation elements 122 and the inner wall of the artery. The renal artery RA thus defines, for the selected size of catheter 12, outer dimension D₂ and pitch HP. Furthermore, when therapeutic assembly 100 deploys into the helical shape within renal artery RA, distal end 118a of therapeutic assembly 100 moves axially towards the proximal end 118b of therapeutic assembly 100 (or vice versa) such that the deployed length L₁ is defined by renal artery RA and is less than the unexpanded or delivery length.

Upon deployment, the pre-formed helical shape of support structure 110 provides proud portions 120 that are offset from the curvilinear axis CA₁ in a direction radially outward from the central helical axis HA₁ and in an orientation toward an interior wall of the target blood vessel. Referring to FIG. 3B, therapeutic assembly 100 is thus configured to press proud portions 120 and neuromodulation elements 122 against an interior wall of a blood vessel for delivering therapeutically effective energy to target tissue (e.g., one or more nerves) of the patient. For example, when therapeutic assembly 100 is deployed in renal artery RA of the patient, the central helical axis HA₁ is generally aligned with the central longitudinal axis LA₂ of renal artery RA such that proud portions 120 can be offset from the pre-formed spiral shape (e.g., the curvilinear axis CA₁) in a direction radially outward from the central longitudinal axis LA₂. Accordingly, offset proud portions 120 are configured to position neuromodulation elements 122 associated with proud portions 120 in stable apposition with the interior wall of renal artery RA. Further, adjacent portions 124, which are collinear with the curvilinear axis CA₁ of the helical shape of the support structure 110 are conversely configured to be radially spaced apart from the interior wall of renal artery RA (e.g., in a direction radially inward toward central longitudinal axis LA₂ with respect to proud portions 120).

The helix-shaped deployed configuration of support structure 110 is further illustrated in FIG. 3C, which is a transverse cross-sectional view of therapeutic assembly 100 along the line 3C-3C of FIG. 3B. Referring to FIGS. 3B and 3C together, the distal portion of support structure 110 defines a helical shape having the plurality of proud portions 120 offset from the spiral shape (e.g., not collinear and/or not concentrically centered with the spiral shape) in a direction radially outward from central longitudinal axis LA₂ such that proud portions 120 provide the plurality of spaced-apart contact regions 123 for contacting an inner wall of the blood vessel (e.g., renal artery RA). In one embodiment, the distal portion of support structure 110 can have a pre-set spiral/helical configuration such that support structure 110 self-expands to a deployed geometry within the renal artery RA. As illustrated in FIGS. 3B and 3C, the spiral/helix defined by the curvilinear axis CA₁ (FIG. 3A) of the support structure 110 has a transverse dimension about the central helical axis HA₁ that is less than a renal artery inner diameter RA_{D1} (FIG. 3C) and a maximum length in the direction of the central longitudinal axis CA₁ that is preferably less than or can be accommodated by the renal artery length (not shown). In one embodiment, proud portions 120 can be offset from the curvilinear axis CA₁ of support structure 110 by less than about 0.5 mm. In other embodiments, the offset can be less than about 0.2 mm. In further embodiments, the offset can be less than 0.1 mm (e.g., 0.05 mm, 0.03 mm, 0.01 mm, etc.). In other embodiments, however, support structure 110 may have a different arrangement and/or different dimensions.

As shown in FIGS. 3A and 3B, proud portions 120 and neuromodulation elements 122 may be distributed on helical support structure 110 in a desired arrangement. For example, the axial distances XX between adjacent proud portions 120 may be selected so that the edges of the lesions formed by individual neuromodulation elements 122 on the inner wall of renal artery RA are overlapping or non-overlapping. Referring to FIG. 3B, axial distance XX may be about 2 mm to about 1 cm. In a particular embodiment, the axial distance XX may be in the range of about 2 mm to about 5 mm. In another embodiment, proud portions 120 may be spaced apart about 30 mm. In still another embodiment, proud portions 120 are spaced apart about 11 mm. In yet another embodiment, proud portions 120 are spaced apart about 17.5 mm.

In some embodiments, proud portions 120 are both longitudinally and circumferentially offset from one another. FIG. 3C, for example, illustrates the circumferential offset or separation of proud portions 120 from one another around the circumference of the deployed spiral support structure 110. The offset angles may be selected such that, when energy is applied to the renal artery via neuromodulation elements 122, the lesions may or may not overlap circumferentially.

In one embodiment, support structure 110 can include a solid structural element, e.g., a wire, tube, coiled or braided cable. Support structure 110 may be formed from biocompatible metals and/or polymers, including polyethylene terephthalate (PET), polyamide, polyimide, polyethylene block amide copolymer, polypropylene, or polyether ether ketone (PEEK) polymers. In some embodiments, components of support structure 110 may be electrically nonconductive, electrically conductive (e.g., stainless steel, nickel-titanium alloy (nitinol), silver, platinum, nickel-cobalt-chromium-molybdenum alloy), or a combination of electrically conductive and nonconductive materials. In one particular embodiment, for example, support structure 110 can include a pre-shaped material, such as spring temper stainless steel or nitinol. Furthermore, in particular embodiments, support structure 110 may be formed, at least in part, from radiopaque materials that are capable of being fluoroscopically imaged to allow a clinician to determine if therapeutic assembly 100 is appropriately placed and/or deployed in the renal artery. Radiopaque materials may include, for example, barium sulfate, bismuth trioxide, bismuth subcarbonate, powdered tungsten, powdered tantalum, or various alloys of certain metals, including gold and platinum, and these materials may be directly incorporated into structural elements or may form a partial or complete coating on support structure 110.

As mentioned above, pre-shaped control member 116 may be used to impart a spiral/helical shape to support structure 110 having spaced-apart proud portions 120 in therapeutic assembly 100. In one embodiment, control member 116 can be a tubular structure comprising a nitinol multifilar stranded wire with a lumen therethrough and sold under the trademark HELICAL HOLLOW STRAND (HHS), and commercially available from Fort Wayne Metals of Fort Wayne, Ind. Control member 116 may be formed from a variety of different types of materials, may be arranged in a single or dual-layer configuration, and may be manufactured with a selected tension, compression, torque and pitch direction. The HHS material, for example, may be cut using a laser, electrical discharge machining (EDM), electrochemical grinding (ECG), or other suitable means to achieve a desired finished component length and geometry.

Forming control member 116 of nitinol multifilar stranded wire(s) or other similar materials is expected to provide a desired level of support and rigidity to the therapeutic assembly 100 without requiring additional reinforcement wire(s) or other reinforcement features within support structure 110. This feature is expected to reduce the number of manufacturing processes required to form therapeutic assembly 100 and reduce the number of materials required for the device.

FIG. 3D is an enlarged view of a portion of support structure 110 of FIG. 3A showing the geometric pre-shaped pattern of control member 116 through a transition between adjacent or interposing portions 124 and a proud portion 120. For example, control member 116 is not collinear with the curvatures of adjacent portions 124 through the transition from the adjacent portions 124 to the proud portion 120. In one embodiment, the pre-formed helical shape having proud portions 120 can be formed from a shape memory material (e.g., (nitinol)) wire or tube that is shaped around a mandrel (not shown) having spaced-apart protrusions on the outer surface of a generally helical shape. The protrusions can form the raised or otherwise offset portions with respect to the primary spiral/helix geometry of control member 116 using conventional shape-setting techniques known in the art. In one specific example, nitinol superelastic wire can typically be heated at approximately 510° C. for approximately 5 minutes followed by a water quench. In another embodiment, a flat sheet of nitinol or other pseudoelastic material can be fabricated into a square wave pattern and further wrapped about a shape rod or mandrel for pre-forming the helical shape having offset proud portions 120.

In yet further embodiments, a stiffness of control member 116, and thereby support structure 110, can vary along the central longitudinal axis LA1 of support structure 110. For example, control member 116 at proud portions 120 can have a first stiffness and at adjacent portions 124 can have a second stiffness greater than the first stiffness. In various embodiments, variable stiffness along portions of control member 116 and/or support structure 110 could be provided using variations in a braid or weave pattern, coiled structures, woven structures and/or wire density as known by one of ordinary skill in the art of fabricating shaped devices. In such arrangements, proud portions 120 can be at least approximately in axial alignment (e.g., the offset resulting in an outer dimension less than 10% greater than the outer dimension D₁) with longitudinal axis LA₁ of support structure 110 in a delivery configuration (e.g., the stiffness of guidewire 50 can be greater than the stiffness of the shape memory material at proud portions 120) such that therapeutic assembly 100 can maintain a low-profile for delivery through a suitably-sized guide catheter (e.g., 6 Fr, 7 Fr, less than 8 Fr, etc.).

In one embodiment, flexible tube 114 provides an insulating layer or sleeve over control member 116 and energy supply wires 121 to further electrically isolate the material (e.g., nitinol) of support structure 110 (e.g., as shown in FIG. 3D). Flexible tube 114 may be composed of a polymer material such as polyamide, polyimide, polyether block amide copolymer sold under the trademark PEBAX, polyethylene terephthalate (PET), polypropylene, an aliphatic, polycarbonate-based thermoplastic polyurethane sold under the trademark CARBOTHANE, or a polyether ether ketone (PEEK) polymer, thermoplastic polyether urethane, other suitable materials or combinations thereof. In one exemplary embodiment, flexible tube 114 comprises an inner layer of PET tubing shrunk around control member 116 and energy supply wires 121, and an outer layer of thermoplastic polyether urethane shrunk around thermocouple wires (not shown) and the inner layer. The material properties and dimensions of tube 114 are selected to provide the necessary flexibility for tube 114 to readily deform between a relaxed, substantially straight shape and a shape that conforms to the spiral/helical deployed shape of control member 116. In other words, tube 114 is more flexible than control member 116 such that the shape of the combined components is defined in large part by the shape of control member 116. In certain embodiments, adjacent portions 124 are covered by the insulative material of flexible tube 114 while neuromodulation elements 122 are not covered by the insulative material.

In one embodiment, control member 116 and inner wall of tube 114 can be in intimate contact with little or no space therebetween (as best seen in FIG. 3D). In some embodiments, for example, tube 114 can have a larger cross-sectional dimension (e.g., diameter) than control member 116 before assembly such that applying heat to tube 114 during the manufacturing process shrinks the tube onto control member 116, as will be understood by those familiar with the ordinary use of shrink tubing materials. This feature is expected to inhibit or eliminate wrinkles or kinks that might occur in tube 114 as therapeutic assembly 100 transforms from the relatively straight delivery configuration (FIG. 2) to the generally spiral/helical deployed configuration (e.g., FIG. 3A).

In other embodiments, control member 116 and/or other components of therapeutic assembly 100 may be composed of different materials and/or have a different arrangement. For example, control member 116 may be formed from other suitable shape memory materials (e.g., wire or tubing besides HHS or Nitinol, super elastic polymers, electro-active polymers) that are pre-formed or pre-shaped into the desired deployed configuration. Alternatively, control member 116 may be formed from multiple materials such as a composite of one or more polymers and metals.

In one embodiment, individual neuromodulation elements 122 can be electrodes configured to deliver energy (e.g., electrical energy, RF energy, pulsed electrical energy, non-pulsed electrical energy, thermal energy, etc.) across the wall of renal artery RA. In a specific embodiment, each neuromodulation element 122 can deliver a thermal RF field to targeted renal nerves adjacent the wall of renal artery RA. Referring to FIGS. 2-3D together, individual neuromodulation elements 122 can include a band electrode surrounding a corresponding one of proud portions 120 of support structure 110 (e.g., over flexible tube 114). For example, neuromodulation element 122 can be a band electrode bonded to tube 114 using an adhesive. Although band or tubular electrodes are illustrated, in other embodiments disc or flat electrodes may also be employed. In still another embodiment, electrodes having a spiral or coil shape may be utilized. Neuromodulation elements 122 may be formed from any suitable metallic material (e.g., gold, platinum, an alloy of platinum and iridium, etc.). In other embodiments, however, the number, arrangement, and/or composition of neuromodulation elements 122 may vary. For example, one or more neuromodulation elements 122 may be placed on support structure 110 at another location proximal to and/or separate from proud portions 120.

Neuromodulation elements 122 are electrically connected to an external energy source (such as energy source 30, FIG. 1) by conductor or bifilar wires (not shown) extending through catheter 12. Neuromodulation elements 122 may be welded or otherwise electrically coupled to their energy supply wire, and the wires can extend the entire length of catheter 12 (e.g. inside, outside or within a wall of catheter 12 and shaft 14) such that a proximal end thereof is coupled to energy source 30 (FIG. 1).

In operation and referring to FIGS. 1-3D together, after the distal portion of support structure 110 is self-expanded or otherwise deployed to its pre-set spiral/helical configuration with proud portions 120 providing contact regions 123 in apposition with the interior wall of the renal artery RA, therapeutically-effective energy can be delivered via neuromodulation elements 122 across the wall of renal artery RA to targeted renal nerves (not shown) at one or more treatment locations.

After forming sufficient lesions or treatment zones to achieve neuromodulation, and in accordance with one method, therapeutic assembly 100 may be transformed back to the low-profile delivery configuration by axially advancing guidewire 50 relative to therapeutic assembly 100 (e.g., within lumen 111 of support structure 110). Once guidewire 50 is in position at the treatment site and therapeutic assembly 100 is in the low-profile delivery configuration, therapeutic assembly 100 can be pulled back with or over guidewire 50.

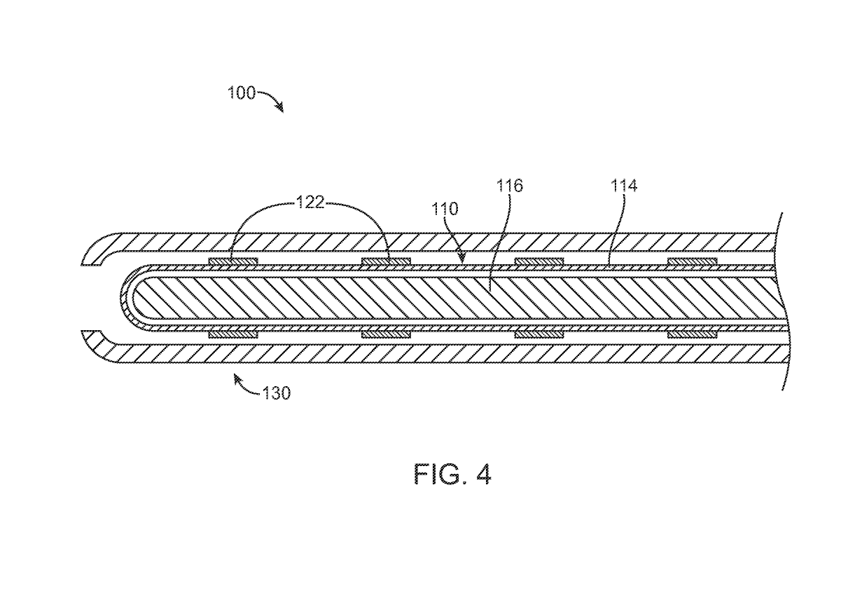

FIG. 4 illustrates another embodiment in which therapeutic assembly 100 is constrained in the delivery configuration (e.g., a generally straight or collapsed configuration) within the lumen of a tubular sheath or delivery element 130. Delivery element 130 can have a suitable radial and bending stiffness for constraining the distal portion of support structure 110 in a generally straight or non-spiral low-profile configuration (e.g., the delivery configuration). In some embodiments, for example, delivery element 130 may comprise a guide catheter or a straightening sheath sized and shaped to restrain one or more components of therapeutic assembly 100 in the low-profile configuration for delivery to the target treatment site within renal artery RA. In the collapsed, low-profile configuration, the geometry of therapeutic assembly 100 is configured to move through delivery element 130 to the treatment site. Persons of skill in the art of catheters will understand that when delivery element 130 is a guide catheter, it will typically have a pre-formed curved region (not shown) near the distal end. Thus, the delivery configuration of therapeutic assembly 100 will be reduced, i.e. “low” in transverse profile, but not necessarily straight as it passes through the curved region of the guide catheter. Therapeutic assembly 100, in the low-profile configuration is sufficiently flexible to pass through the guide catheter, including the curved region. In some embodiments, delivery element 130 may be 8 Fr or smaller (e.g., a 6 Fr guide catheter) to accommodate small (e.g., 3-4 mm diameter) renal arteries during delivery of therapeutic assembly 100 to the treatment site. In other embodiments, however, delivery element 130 may have a different size.

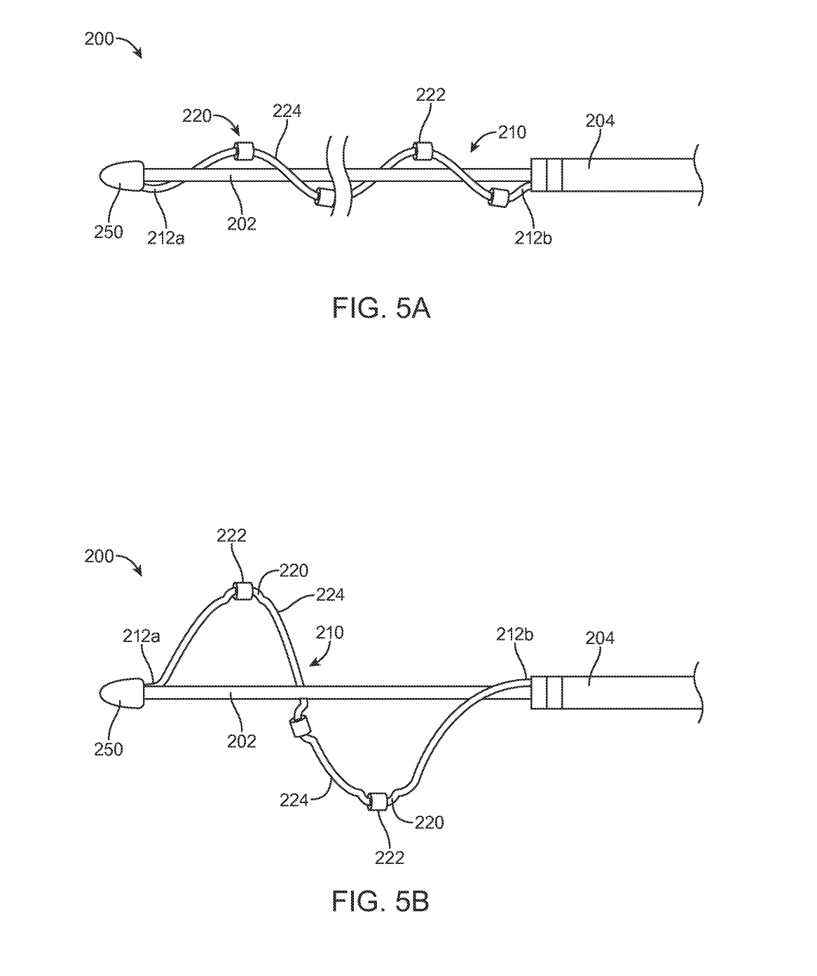

FIGS. 5A and 5B, illustrate a distal portion of a therapeutic assembly 200 configured in accordance with further embodiments of the present technology. More specifically, FIGS. 5A and 5B illustrate a therapeutic assembly 200 having a tubular support structure 210 helically wrapped about a control member 202. Support structure 210 can include a number of features generally similar to support structure 110 described above. For example, support structure 210 can include a shape-memory or other material that is pre-formed and expands when not constrained. In one embodiment, support structure 210 is configured to have a plurality of spaced-apart proud portions 220 that are offset with respect to a pre-formed spiral shape of deployed support structure 210. Therapeutic assembly 200 can further include a plurality of neuromodulation elements 222 disposed about the support structure 210 at proud portions 220.

In the illustrated embodiment, the therapeutic assembly 200 further includes a control member 202 and an end piece, such as a tip 250, coupled to a distal region or portion 212a of support structure 210 and control member 202. Tip 250 can have a conical or bullet shape. For example, tip 250 can include a rounded distal portion for atraumatic insertion of therapeutic assembly 100 into a target blood vessel. In other embodiments, the end piece can be a collar or other type of cap. A proximal region or portion 212b of support structure 210 is coupled to and affixed to an elongated shaft 204 of the therapeutic assembly 200. Elongated shaft 204 defines a central passageway for passage of control member 202. Control member 202 may be, for example, a solid wire made from a metal or polymer. Control member 202 extends from elongated shaft 204 and is affixed to tip 250. Moreover, control member 202 slidably passes through elongated shaft 204 to an actuator in a handle assembly (not shown).

In this embodiment, control member 202 is configured to move distally and proximally through elongated shaft 204 so as to move tip 250 and distal region 212a of support structure 210 accordingly. Distal and proximal movement of distal region 212a respectively lengthens and shortens the axial length of the helix of support structure 210 so as to transform therapeutic assembly 100 between a delivery configuration (FIG. 5A) and a deployed configuration (FIG. 5B) such that proud portions 220 carrying neuromodulation elements 222 engage the interior wall of the target blood vessel (not shown) while adjacent or interposing portions 224 separating proud portions 220 are configured to be spaced apart from the interior wall of the target blood vessel (not shown).

### Selected Examples of Methods for Delivery and Deployment of Therapeutic Assemblies

Several suitable delivery methods are disclosed herein and discussed further below; however, one of ordinary skill in the art will recognize a plurality of methods suitable to deliver therapeutic assembly 100 to the treatment site and to deploy support structure 110 from the delivery configuration to the deployed configuration.

FIG. 6 (with additional reference to FIG. 1) illustrates at least one step of modulating renal nerves with an embodiment of system 10. Therapeutic assembly 100 is shown positioned within the renal plexus RP and catheter 12 is shown in an intravascular path P extending from a percutaneous access site in a femoral (illustrated), brachial, radial, or axillary artery to a targeted treatment site within a respective renal artery RA. As illustrated, a section of proximal portion 16 of catheter shaft 14 is exposed externally of the patient even as therapeutic assembly 100 has been advanced fully to the targeted treatment site in the patient. By manipulating proximal portion 16 of shaft 14 from outside intravascular path P, the clinician may advance shaft 14 through the sometimes tortuous intravascular path P and remotely manipulate distal portion 20 of shaft 14.

In the method step illustrated in FIG. 6, therapeutic assembly 100 extends intravascularly to the treatment site over guidewire 50 using an OTW technique. As noted previously, the distal end of therapeutic assembly 100 may define a lumen or passageway for receiving guidewire 50 for delivery of catheter 12 using either OTW or RX techniques. At the treatment site, guidewire 50 can be at least partially axially withdrawn or removed, and therapeutic assembly 100 can transform or otherwise be converted to a deployed arrangement for delivering energy at the treatment site as described above with respect to FIGS. 2-5B. Guidewire 50 may comprise any suitable medical guidewire sized to slidably fit within lumen 111 of catheter 12. In one particular embodiment, for example, guidewire 50 may have a diameter of 0.356 mm (0.014 inch). In other embodiments, therapeutic assembly 100 may be delivered to the treatment site within a guide sheath (not shown) with or without using guidewire 50. When therapeutic assembly 100 is at the target site, the guide sheath may be at least partially withdrawn or retracted and therapeutic assembly 100 can be transformed into the deployed configuration. In still other embodiments, shaft 14 may be steerable itself such that therapeutic assembly 100 may be delivered to the treatment site without the aid of guidewire 50 and/or guide sheath.

Image guidance, e.g., computed tomography (CT), fluoroscopy, intravascular ultrasound (IVUS), optical coherence tomography (OCT), or another suitable guidance modality, or combinations thereof, may be used to aid the clinician's positioning and manipulation of therapeutic assembly 100. For example, a fluoroscopy system (e.g., including a flat-panel detector, x-ray, or c-arm) can be utilized to accurately visualize and identify the target treatment site. In other embodiments, the treatment site can be determined using IVUS, OCT, and/or other suitable image mapping modalities that can correlate the target treatment site with an identifiable anatomical structure (e.g., a spinal feature) and/or a radiopaque ruler (e.g., positioned under or on the patient) before delivering catheter 12 and/or therapeutic assembly 100. Further, in some embodiments, image guidance components (e.g., IVUS, OCT) may be integrated with catheter 12, support structure 110 and/or run in parallel with catheter 12 to provide image guidance during positioning and removal of therapeutic assembly 100. For example, image guidance components (e.g., IVUS or OCT) can be coupled to at least one of therapeutic assembly 100 to provide three-dimensional images of the vasculature proximate the target site to facilitate positioning or deploying therapeutic assembly 100 within the target renal blood vessel.

Referring to FIGS. 1-6 together, the purposeful application of energy from neuromodulation elements 122 (e.g., carried by proud portions 120) may be applied to target tissue to induce one or more desired neuromodulating effects on localized regions of the renal artery and adjacent regions of the renal plexus RP, which lay intimately within, adjacent to, or in close proximity to the adventitia of renal artery RA. The purposeful application of the energy may achieve neuromodulation along all or at least a portion of renal plexus RP. The neuromodulating effects are generally a function of, at least in part, power, time, contact between neuromodulation elements 122 (FIGS. 3B and 3C) and the vessel wall, and blood flow through the vessel. The neuromodulating effects may include denervation, thermal ablation, and/or non-ablative thermal alteration or damage (e.g., via sustained heating and/or resistive heating). Desired thermal heating effects may include raising the temperature of target neural fibers above a desired threshold to achieve non-ablative thermal alteration, or above a higher temperature to achieve ablative thermal alteration. For example, the target temperature may be above body temperature (e.g., approximately 37° C.) but less than about 45° C. for non-ablative thermal alteration, or the target temperature may be about 45° C. or higher for the ablative thermal alteration. Desired non-thermal neuromodulation effects may include altering the electrical signals transmitted in a nerve.

In operation (and with reference to FIGS. 1-6), after being positioned at a desired location within renal artery RA of the patient, therapeutic assembly 100 may be transformed from its delivery configuration (e.g., shown in FIG. 2) to its deployed configuration (e.g., shown in FIGS. 3A-3C). The transformation may be initiated using an arrangement of device components as described herein with respect to the particular embodiments and their various modes of deployment. In one embodiment, for example, therapeutic assembly 100 may be deployed by retracting guidewire 50 until the pre-formed spiral shape of support structure 110 provides a shape-recovery force sufficient to overcome the straightening force provided by distalmost portion 52 of guidewire 50. After treatment, therapeutic assembly 100 may be transformed back to the low-profile delivery configuration by axially advancing guidewire 50 relative to therapeutic assembly 100.

### Additional Embodiments

Features of the catheter device components described above and illustrated in FIGS. 1-6 can be modified to form additional embodiments configured in accordance with the present technology. For example, neuromodulation system 10 can provide delivery of any of therapeutic assemblies 100 illustrated in FIGS. 2-5B using one or more additional delivery elements such as guide catheters, straightening sheaths, and/or guidewires. Similarly, the therapeutic assemblies described above and illustrated in FIGS. 1-5B showing neuromodulation elements at the proud portions can also include additional electrode elements, wires, and energy delivery features positioned along the treatment device.

Various method steps described above for delivery and deployment of the therapeutic assembly components also can be interchanged to form additional embodiments of the present technology. For example, while the method steps described above are presented in a given order, alternative embodiments may perform steps in a different order. The various embodiments described herein may also be combined to provide further embodiments.

### Renal Neuromodulation

Renal neuromodulation is the partial or complete incapacitation or other effective disruption of nerves innervating the kidneys. In particular, renal neuromodulation comprises inhibiting, reducing, and/or blocking neural communication along neural fibers (i.e., efferent and/or afferent nerve fibers) innervating the kidneys. Such incapacitation can be long-term (e.g., permanent or for periods of months, years, or decades) or short-term (e.g., for periods of minutes, hours, days, or weeks). Renal neuromodulation is expected to efficaciously treat several clinical conditions characterized by increased overall sympathetic activity, and in particular conditions associated with central sympathetic over-stimulation such as hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, osteoporosis, and sudden death. The reduction of afferent neural signals contributes to the systemic reduction of sympathetic tone/drive, and renal neuromodulation is expected to be useful in treating several conditions associated with systemic sympathetic over activity or hyperactivity. Renal neuromodulation can potentially benefit a variety of organs and bodily structures innervated by sympathetic nerves.

Various techniques can be used to partially or completely incapacitate neural pathways, such as those innervating the kidney. The purposeful application of energy (e.g., electrical energy, thermal energy) to tissue by energy delivery element(s) or components such as those described in conjunction with the intravascular treatment assemblies above, can induce one or more desired thermal heating effects on localized regions of the renal artery and adjacent regions of the renal plexus, which lay intimately within or adjacent to the adventitia of the renal artery. The purposeful application of the thermal heating effects can achieve neuromodulation along all or a portion of the renal plexus.

The thermal heating effects can include both thermal ablation and non-ablative thermal alteration or damage (e.g., via sustained heating and/or resistive heating). Desired thermal heating effects may include raising the temperature of target neural fibers above a desired threshold to achieve non-ablative thermal alteration, or above a higher temperature to achieve ablative thermal alteration. For example, the target temperature can be above body temperature (e.g., approximately 37° C.) but less than about 45° C. for non-ablative thermal alteration, or the target temperature can be about 45° C. or higher for the ablative thermal alteration.

More specifically, exposure to thermal energy (heat) in excess of a body temperature of about 37° C., but below a temperature of about 45° C., may induce thermal alteration via moderate heating of the target neural fibers or of vascular structures that perfuse the target fibers. In cases where vascular structures are affected, the target neural fibers are denied perfusion resulting in necrosis of the neural tissue. For example, this may induce non-ablative thermal alteration in the fibers or structures. Exposure to heat above a temperature of about 45° C., or above about 60° C., may induce thermal alteration via substantial heating of the fibers or structures. For example, such higher temperatures may thermally ablate the target neural fibers or the vascular structures. In some patients, it may be desirable to achieve temperatures that thermally ablate the target neural fibers or the vascular structures, but that are less than about 90° C., or less than about 85° C., or less than about 80° C., and/or less than about 75° C. Regardless of the type of heat exposure utilized to induce the thermal neuromodulation, a reduction in renal sympathetic nerve activity (RSNA) is expected.

### CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/we claim:

1. A neuromodulation catheter, comprising:
an elongated shaft; and
a therapeutic assembly disposed at a distal portion of the elongated shaft and adapted to be located at a target location within a target blood vessel of a human patient, the therapeutic assembly including—
a tubular support structure comprising a pre-formed helical shape having a plurality of proud portions longitudinally separated by interposing portions, wherein the proud portions are offset with respect to the pre-formed helical shape; and
a plurality of energy delivery elements, each element being carried by the support structure at a corresponding proud portion,
wherein the elongated shaft and the therapeutic assembly together define a guidewire lumen configured to slidably receive a guidewire therethrough,
wherein at least partial removal of the guidewire relative to the therapeutic assembly transforms the support structure from a low-profile delivery configuration to a deployed configuration defined by the pre-formed helical shape of the support structure, and
wherein, when the support structure is in the deployed configuration, the proud portions are configured to position the energy delivery elements in apposition with an inner wall of the target blood vessel.

2. The neuromodulation catheter of claim 1, further comprising insulative material about the support structure and associated with at least the interposing portions.

3. The neuromodulation catheter of claim 2 wherein the insulative material comprises polyethylene terephthalate (PET) heat shrink tubing.

4. The neuromodulation catheter of claim 1 wherein, when the support structure is in the delivery configuration, the proud portions are spaced apart from each other along a central longitudinal axis of the support structure and the proud portions are at least approximately co-axial with the central longitudinal axis.

5. The neuromodulation catheter of claim 1 wherein the proud portions are offset with respect to the pre-formed helical shape by a dimension sufficient to cause the interposing portions be radially spaced apart from the inner wall of the target blood vessel when the support structure is in the vessel in the deployed configuration.

6. The neuromodulation catheter of claim 1 wherein each energy delivery element comprises a band electrode.

7. The neuromodulation catheter of claim 1 wherein the support structure comprises a nitinol multifilar stranded wire.

8. The neuromodulation catheter of claim 1 wherein a stiffness of the support structure varies along the length of the support structure, and wherein the proud portions have a first stiffness and the interposing portions have a second stiffness greater than the first stiffness.

9. The neuromodulation catheter of claim 1 wherein, when the support structure is in the deployed configuration, the proud portions are not collinear with a curvilinear axis of the pre-formed helical shape.

10. The neuromodulation catheter of claim 1 wherein:
the support structure has a shape-recovery force insufficient to overcome a straightening force provided by a distal region of the guidewire when the guidewire is within the guidewire lumen of the therapeutic assembly;
the support structure is configured to transform to the deployed configuration when the distal region of the guidewire is withdrawn through the guidewire lumen to a point proximal of the therapeutic assembly; and
the proud portions are offset with respect to the pre-formed helical shape in a direction radially outward from a central axis of the helical shape when the support structure is in the deployed configuration.

11. A neuromodulation assembly adapted for delivery into a target blood vessel and configured to deliver radiofrequency (RF) energy to target tissue of a human patient, wherein the neuromodulation assembly is carried at a distal end of a catheter and is transformable between a low-profile delivery configuration and a radially expanded deployed configuration, the neuromodulation assembly comprising:
a support structure having a pre-formed helical shape with a plurality of spaced apart steps, the steps configured to be in apposition with an inner wall of the target blood vessel when the assembly is in the deployed configuration, wherein—
the support structure is tubular and has a substantially uniform outer dimension along a length thereof, and
the steps are out of axial alignment with a curvilinear axis of the helical shape when the neuromodulation assembly is in the deployed configuration; and
a plurality of neuromodulation elements, wherein individual neuromodulation elements are positioned at a corresponding steps and are configured to deliver the RF energy to target tissue when the steps are in apposition with the inner wall of the target blood vessel.

12. The neuromodulation assembly of claim 11 wherein the neuromodulation element comprises a band electrode disposed about the outer dimension of the support structure at the individual steps.

13. The neuromodulation assembly of claim 11 wherein the support structure comprises a nitinol multifilar stranded wire that is constrained in a relatively straight configuration when the neuromodulation assembly is in the delivery configuration.

14. The neuromodulation assembly of claim 11 wherein the support structure comprises a guidewire lumen configured to slidably receive a guidewire therethrough, and wherein the support structure has a shape-recovery force insufficient to overcome a straightening force provided by a distal region of the guidewire when the guidewire is within the guidewire lumen.

15. The neuromodulation assembly of claim 11 wherein, in the deployed configuration, the steps are configured to protrude toward and to contact the inner wall of the target blood vessel such that interposing segments of the support structure are radially spaced apart from the inner wall of the target blood vessel.

16. The neuromodulation assembly of claim 15 wherein a stiffness of the support structure varies along a length of the support structure, and wherein the steps have a first stiffness and the interposing segments have a second stiffness greater than the first stiffness.

17. The neuromodulation assembly of claim 11, further comprising an insulative sleeve disposed about at least a portion of the support structure, wherein the individual neuromodulation elements are not covered by the insulative sleeve.

18. A neuromodulation system for treatment of a human patient, the system comprising:
an electric field generator configured to deliver radiofrequency (RF) energy to target tissue of a human patient;
a catheter having a proximal portion and distal portion, wherein the distal portion of the catheter is configured for intravascular delivery to a blood vessel of the patient;
a treatment assembly disposed at the distal portion of the catheter, wherein the treatment assembly is selectively transformable between a unexpanded configuration and a radially expanded configuration having a generally helical structure, and wherein the generally helical structure includes a plurality of spaced apart contact regions for contacting an inner wall of the blood vessel; and
a plurality of electrodes carried by the spiral structure at the contact regions, wherein the electrodes are configured to deliver RF energy from the electric field generator to the inner wall of the blood vessel,
wherein, in the radially expanded configuration, the contact regions are spaced apart from each other along a central axis of the generally helical structure, and wherein the contact regions project radially away from the generally helical structure without protruding toward the central axis of the helical structure.

19. A method of performing neuromodulation within a target blood vessel of a human patient, the method comprising:
intravascularly delivering a neuromodulation catheter in a low-profile delivery configuration to a target treatment site within the target blood vessel, wherein the neuromodulation catheter comprises—
an elongated shaft; and
a multi-electrode array disposed at a distal portion of the shaft and composed, at least in part, of a tubular structure having a generally constant outer dimension and formed of multifilar nitinol wire;
transforming the neuromodulation catheter from the low-profile delivery configuration to a deployed configuration, wherein the tubular structure has a radially expanded, generally helical shape having a plurality of spaced-apart proud portions, and wherein the individual proud portions are associated with an electrode of the multi-electrode array, and further wherein each electrode associated with an individual proud portion is configured to contact an inner wall of the renal blood vessel; and
selectively delivering energy to one or more of the electrodes of the multi-electrode array to modulate target nerves proximate to the inner wall of the target blood vessel.

20. The method of claim 19 wherein, when the neuromodulation catheter is in the deployed configuration, the helical shape has a curvilinear axis and the proud portions are out of axial alignment with the curvilinear axis of the helical shape.

21. The method of claim 19 wherein:
intravascularly delivering a neuromodulation catheter includes delivering the neuromodulation catheter over a guidewire; and
transforming the neuromodulation catheter from the low-profile delivery configuration to a deployed configuration includes withdrawing the guidewire in a proximal direction until the neuromodulation catheter transforms from the low-profile delivery configuration to the deployed configuration.

22. The method of claim 19, further comprising:
transforming the neuromodulation catheter from the deployed configuration to the delivery configuration after selectively delivering energy; and
removing the neuromodulation catheter from the patient.

23. The method of claim 19 wherein:
intravascularly delivering the neuromodulation catheter includes delivering the multi-electrode array through a guide catheter, wherein the guide catheter is configured to constrain the neuromodulation catheter in the delivery configuration; and
transforming the neuromodulation catheter from the delivery configuration to a deployed configuration comprises withdrawing the guide catheter in a proximal direction until the neuromodulation catheter recovers from the low-profile delivery configuration to the deployed configuration within the target blood vessel.
